(19) United States
(12) Patent Application Publication
(10) Pub. No.: US 2024/0398622 A1
(43) Pub. Date: Dec. 5, 2024

(54) PLANNING DEVICE FOR GENERATING CONTROL DATA FOR A LASER DEVICE OF A TREATMENT APPARATUS FOR REFRACTIVE CORRECTION OF AN EYE, TREATMENT APPARATUS, METHOD FOR GENERATING CONTROL DATA AND METHOD FOR REFRACTIVE CORRECTION

(71) Applicant: Carl Zeiss Meditec AG, Jena (DE)

(72) Inventor: Marcel DUSI, Saalburg-Ebersdorf (DE)

(21) Appl. No.: 18/698,824

(22) PCT Filed: Oct. 4, 2022
(86) PCT No.: PCT/EP2022/077569

§ 371 Date: Apr. 5, 2024

(30) Foreign Application Priority Data
Oct. 5, 2021 (DE) 10 2021 125 782.4

(51) Int. Cl. A61F 9/008

(52) U.S. Cl. CPC A61F 9/00827; A61F 2009/00872; A61F 2009/00878

(57) ABSTRACT

The invention relates to a planning device for generating control data for a laser device of a treatment apparatus for refractive correction of an eye. The treatment apparatus (10) has a laser device (20) for modifying the cornea of the eye A by irradiating it with a pulsed laser beam (22), and a control device (30) for controlling the laser device. The control device is designed to control the laser device with a view to focusing the laser beam on target points of the cornea and modifying the cornea by means of photodisruption. The planning device is designed to determine at least one three-dimensional pattern of target points of the cornea, wherein the intrastromal photodisruption of a region of the cornea predefined by the pattern brings about a refractive correction of the eye treated by a pretreatment. A treatment apparatus, a method for generating control data, a method for refractive correction of an eye, and computer program products are also provided.

### CLAIM TO PRIORITY

This application is a United States National Stage entry of Application PCT/EP2022/077569, filed Oct. 4, 2022, which claims priority to DE Patent Application No 10 2021 125 782.4, filed Oct. 5, 2021, each of which are incorporated by reference in their entireties in this application.

### TECHNICAL FIELD

Example embodiments of the invention relate to a planning device for generating control data for a laser device of a treatment apparatus for refractive correction of an eye after a pretreatment or for refractive correction of an eye that includes a pretreatment, to a treatment apparatus for refractive correction of an eye after a pretreatment or for refractive correction of an eye that includes a pretreatment, to a method for generating control data for a laser device of a corresponding treatment apparatus, to a method for refractive correction of an eye after a pretreatment or for refractive correction of an eye that includes a pretreatment, and to computer program products.

### BACKGROUND

Attachment lenses in the form of spectacles have been used since time immemorial to correct human refractive errors. Recent times have seen various approaches of correcting the refractive error of the eye by virtue of modifying the cornea. The modification is intended to ensure a change in the curvature of the cornea. The corneal front surface must be flattened for the purpose of correcting myopia, which is why the corresponding volume of tissue to be removed is thicker in the middle, which is to say in the region of the visual axis, than at the edge. By contrast, the front surface of the cornea must be curved more strongly in order to correct hyperopia, which is why the volume to be removed is thicker at the edge than in the middle. As a result, the overall imaging properties of the eye are influenced so that a refractive error is reduced or, in the ideal case, even entirely compensated for.

A very successful method in this respect was developed by Carl Zeiss Meditec AG and called SMILE. It uses pulsed laser radiation to isolate a lenticule in the cornea, which can then be removed from the cornea through a lateral opening cut which leads to the corneal surface of the eye and serves as a work channel. The volume of the lenticule is structured and dimensioned such that the front surface of the cornea changes its curvature as required for the correction. The method brings about subtractive correction since volume is removed.

In the case of laser in-situ keratomileusis, also called LASIK, a corneal lamella is initially detached from the corneal surface on one side and folded to the side. Afterward, the corneal tissue then exposed is removed by ablation by application of an excimer laser. Once volume lying in the cornea has been evaporated in this manner, the corneal lamella is folded back onto its original location again.

For modification of the cornea, the laser radiation is applied in a pulsed manner, the pulse length generally being less than 1 ps. As a result, the power density for the respective pulse that is required to trigger an optical breakdown is attained in a confined spatial region. A high level of focusing of the laser beam in combination with the short pulses mentioned thus allows the optical breakdown to be used with pinpoint accuracy in the cornea.

During a modification of the cornea by application of laser radiation, multiple processes that are initiated by the pulsed laser radiation proceed in temporal succession in the tissue. If the power density of the radiation in a pulse exceeds a threshold value, an optical breakdown occurs which produces in the cornea a plasma bubble that includes a gas volume. The plasma bubble grows after the optical breakdown has arisen as a result of expanding gases of the plasma. Plasma bubbles of a plurality of laser pulses may coalesce.

However, known laser methods for refractive corneal surgery such as photorefractive keratectomy (PRK), LASIK and SMILE include surgical manipulation of the cornea (PRK, LASIK, SMILE), exposure of stroma for correction purposes (PRK, LASIK), incisions in the cornea (LASIK, SMILE), weakening or removal of corneal tissue that is necessary in addition to refractive correction (PRK, LASIK, SMILE), or a limited possibility for corneal posttreatment by virtue of a permanent lamellar separating plane that arises (LASIK, SMILE).

Also known is an intrastromally focused and short-pulsed laser application resulting in a locally arising photodisruption of corneal material. The resultant change in volume of the stromal tissue produces a change in the corneal (also local) curvature and hence a change in refractive power of the cornea and the entire eye. However, a refractively effective change in volume of the cornea necessitates very many photodisruption events, i.e. single pulses of the laser, in order to evaporate enough corneal material, which then leads to the change in curvature. In order to achieve a refractive change of one diopter in the optical zone of 6.5 mm of the cornea, approximately 250 nl need to be removed (Munnerlyn).

### SUMMARY OF THE INVENTION

One example embodiment of the invention relates to a planning device that generates control data for a laser device of a treatment apparatus for refractive correction of an eye after a pretreatment, in particular after a refractive pretreatment, or for refractive correction of an eye that includes a pretreatment; wherein the laser device is configured to modify the cornea of the eye by radiating in a pulsed laser beam and the treatment apparatus comprises a control device for controlling the laser device; and wherein the control device is configured to control the laser device to focus the laser beam on target points of the cornea and to modify the cornea by application of photodisruption; wherein the planning device is configured to determine at least one three-dimensional pattern of target points of the cornea which brings about a refractive correction of the eye treated by the pretreatment by virtue of the intrastromal photodisruption of a region of the cornea predefined by the pattern and by virtue of a change in volume of the cornea that results from said intrastromal photodisruption.

In all the example embodiments and modifications thereof, the refractive correction of the eye after the pretreatment and/or the refractive correction of an eye that includes a pretreatment are/is a postcorrection of the respective pretreatment. The pretreatment may be a refractive pretreatment. The change in volume of the cornea, for example the stromal tissue, that results from the intrastromal photodisruption leads to the refractive correction of the eye treated by the pretreatment. This makes it possible for the refractive correction of the eye, i.e. the postcorrection, to be brought about only by virtue of the intrastromal photodisruption of the region of the cornea predefined by the pattern and only by virtue of the change in volume of the cornea that results from said intrastromal photodisruption. The three-dimensional pattern of target points is determined for example in such a way that the intrastromal photodisruption does not lead to the production of cuts in the cornea. The change in volume of the cornea that takes place in the context of the postcorrection can therefore be effected without the production of cuts in the cornea and/or without separation of parts of tissue of the eye.

In all the example embodiments and modifications thereof, the intrastromal photodisruption of the region of the cornea predefined by the pattern can bring about a refractive correction of more than 0 and less than 1.5 diopters, for example 0.01 to 1.2 diopters, in another example 0.1 to 1 diopter. In all the example embodiments and modifications thereof, the intrastromal photodisruption of the region of the cornea predefined by the pattern makes it possible to change, for example remove, a volume of approximately 1 to 375 nl, in another example 2.5 to 300 nl, in a further example 25 to 250 nl, of the cornea (Munnerlyn).

The planning device can be configured to determine, for the refractive correction of the eye that includes the pretreatment, at least one interface pattern of target points of the cornea which, in the pretreatment of the cornea, for example by way of the intrastromal photodisruption of a region of the cornea predefined by the interface pattern, brings about the production of at least one interface between parts of the cornea for absorbing a gas volume that arises as a result of photodisruption. The aim here is to produce a state in the cornea which corresponds to a state after a refractive pretreatment carried out a long time before, for example by means of a SMILE treatment in which the eye is refractively corrected by the production of a lenticule within the cornea and the subsequent removal thereof through a narrow opening. The planning device can furthermore be configured to determine, for the refractive correction of the eye after the pretreatment, the pattern of target points in such a way that, during the refractive correction, it causes a gas volume that arises as a result of photodisruption to be absorbed in at least one interface produced in the pretreatment.

In all the example embodiments and modifications thereof, the interface produced in the pretreatment can be formed in the volume of the cornea, for example in the volume of the stroma, without a side cut for guiding a gas volume out of the eye. In all the embodiments and modifications thereof, the gas volume absorbed in the interface produced by the pretreatment can remain at least partly in the cornea.

The planning device can be configured to determine the pattern of target points in such a way that it brings about a refractive correction of the eye treated by the pretreatment of more than 0 and less than 1.5 diopters, for example 0.01 to 1.2 diopters, in another example 0.1 to 1 diopter. In some embodiments, determining at least one three-dimensional pattern of target points of the cornea includes determining a position of the pattern in the cornea and/or determining a shape of the pattern which bring about the refractive correction of the eye treated by the pretreatment.

The pretreatment can include removal of parts of the cornea of the eye by way of producing an interface between parts of the cornea. The planning device can furthermore be configured to calculate control data for the laser device and/or to communicate them to the control device. The planning device can additionally be configured, when determining the pattern of target points, to choose a pattern from a plurality of predetermined patterns, to receive one or more predetermined patterns and/or to use and/or to calculate the pattern as part of control data. The planning device can comprise a memory unit in which e.g. a plurality of predetermined patterns of target points are stored. The planning device can be connected to the control device of the treatment apparatus in a data-conducting manner, for example via interfaces, and/or can be contained in the control device.

The planning device and/or a treatment apparatus comprising the planning device enable(s) an intrastromally focused and short-pulsed laser application after a pretreatment of the cornea, wherein the laser application leads to a locally arising photodisruption of corneal material. After the pretreatment, this enables an additional refractive correction of the eye with a change in volume of the stromal tissue for the purpose of changing the local corneal, curvature and a change in refractive power of the cornea and the entire eye. Refractive errors persisting after refractive interventions are often less than 1.5 diopters spherical equivalent. Using the planning device and/or by application of the treatment apparatus comprising the planning device, such errors can thus be corrected without surgical or mechanical manipulation of the cornea, without exposure of stroma, without incisions in the cornea and without weakening or removal of corneal tissue in addition to the corneal tissue to be removed for the correction. The planning device and/or the treatment apparatus comprising the planning device additionally afford(s) a possibility for corneal treatment after previous or initial production of an interface between parts of the cornea in a pretreatment, such as a permanent lamellar separating plane, for example by way of a pretreatment by application of photodisruption, a refractive pretreatment, a lenticule extraction, LASIK and/or SMILE. Furthermore, the planning device and/or the treatment apparatus comprising the planning device make(s) it possible for the refractive correction to be effected intrastromally, and thus sterilely and noninvasively, and to be repeatable. By way of example, the refractive correction is repeatable even after a loss of suction, i.e. a loss of a negative pressure that can be applied between eye and laser device, for example a contact glass of the laser device, for fixation of the eye. The repeatability is based on the advantage of the embodiments described here that the laser-treated regions of the eye need not be joined together, but rather can also be introduced into the cornea by laser treatment partially and independently of one another. Furthermore, only a small amount of tissue is removed and a small biomechanical change in the cornea takes place.

The planning device can additionally be configured to determine a three-dimensional shape of the pattern of target points which causes at least one part of a gas volume that arises as a result of the photodisruption to be guided away through micro-perforations in the cornea. What can be achieved as a result is that the gas volume is guided away from the cornea through micro-perforations in the cornea toward an edge of a contact glass and thus toward the surroundings.

The planning device can furthermore be configured to determine the pattern of target points in a position adjacent to or adjoining the interface between parts of the cornea which is produced by the pretreatment. The planning device can be configured to determine a three-dimensional shape of the pattern of target points which causes at least one part of a gas volume that arises as a result of the photodisruption to be guided away into the interface. By way of example, it is possible to determine and/or produce a guided drain channel from the refractive correction region of the cornea produced by intrastromal photodisruption to the interface. Furthermore, the planning device can determine a position of the pattern directly over the interface, such that the intrastromal photodisruption, also called intrastromal ablation, takes place directly over the interface and the gas volume produced is introduced there. The interface can be a permanent lamellar separating plane produced by the pretreatment, such as e.g. a pocket produced by a lenticule extraction. Such an interface has a sufficient absorption capacity for the gas volume produced by the photodisruption. The planning device can additionally comprise an interface for receiving data about the pretreatment, for example about the interface between parts of the cornea which is produced by the pretreatment. By way of example, the data about the interface originate from the planning of the pretreatment and/or from examinations of the eye.

Guiding away the gas that arises makes it possible to prevent an obstruction of the laser radiation of subsequent laser pulses and an accumulation of gas centrally in the patient's cornea.

The interface can be produced by a pretreatment by application of photodisruption, a refractive pretreatment, by a lenticule extraction pretreatment, a LASIK pretreatment and/or a SMILE pretreatment. Furthermore, the planning device can be configured to determine the pattern of target points in a position anterior to the interface. As a result, the gas volume produced as a result of the laser process, i.e. the photodisruption, accumulates posterior to the laser radiation and does not obstruct the laser transmission of succeeding laser pulses.

In one example modification, the planning device can be configured to determine the pattern of target points with a plurality of circular planes of target points arranged one over another and/or with a plurality of ring-shaped series of target points. The ring-shaped series can be determined in the shape of a spiral.

The planning device can moreover be configured to determine the circular planes of target points arranged one over another with a position in the cornea, a shape and a number which bring about the refractive correction of the eye treated by the pretreatment. A locally varying change in curvature and therefore refractive power of the cornea can be brought about as a result. The planning device can be configured to determine the pattern with a plurality of ring-shaped series of target points arranged next to one another which form a first connection structure to the interface. The planning device can furthermore be configured to determine the pattern with a plurality of ring-shaped series of target points arranged next to one another which form a second connection structure between the circular planes of target points and/or to the first connection structure to the interface.

In a further modification, the planning device can be configured to determine the control data for the laser device in such a way that, in order to form the first connection structure, the ring-shaped series of target points are produced in an order from posterior to the interface to anterior to the interface. Furthermore, the planning device can be configured to determine the control data for the laser device in such a way that the plurality of circular planes of target points arranged one over another are produced in an order proceeding from the plane closest to the interface. The planning device can be configured to determine the control data for the laser device in such a way that the position, the shape and/or the number of circular planes of target points arranged one over another are produced by blocking out laser pulses.

A further embodiment specifies a control device for a treatment apparatus for refractive correction of an eye after a pretreatment, for example after a refractive pretreatment, or for refractive correction of an eye that includes a pretreatment, wherein the control device comprises a planning device in accordance with embodiments and/or is connected to such a device in a data-conducting manner.

One embodiment relates to a treatment apparatus for refractive correction of an eye after a pretreatment, for example after a refractive pretreatment, or for refractive correction of an eye that includes a pretreatment; wherein the pretreatment includes for example removal of parts of the cornea of the eye by way of producing an interface between parts of the cornea; wherein the treatment apparatus comprises a laser device for modifying the cornea of the eye by radiating in a pulsed laser beam and a control device for controlling the laser device; and wherein the control device is configured to control the laser device for focusing the laser beam on target points of the cornea and for modifying the cornea by application of photodisruption; wherein the treatment apparatus, for example the control device, comprises a planning device in accordance with one of the embodiments above.

A further embodiment of the invention relates to a method for generating control data for a laser device of a treatment apparatus for refractive correction of an eye after a pretreatment, for example after a refractive pretreatment, or for refractive correction of an eye that includes a pretreatment; for example for generating control data for a laser device of a treatment apparatus in accordance with one of the embodiments above; for example using a planning device in accordance with one of the embodiments above; wherein the pretreatment includes for example removal of parts of the cornea of the eye by way of producing at least one interface between parts of the cornea. The laser device is intended for modifying the cornea of the eye by radiating in a pulsed laser beam, for focusing the laser beam on target points of the cornea and for modifying the cornea by application of photodisruption. In this case, control data for the laser device are calculated which predefine at least one three-dimensional pattern of target points of the cornea in such a way that a refractive correction of the eye treated by the pretreatment is brought about by virtue of the intrastromal photodisruption of a region of the cornea predefined by the pattern and by virtue of a change in volume of the cornea that results from said intrastromal photodisruption.

In the method for generating control data, control data for the refractive correction of the eye that includes the pretreatment can be calculated which predefine at least one interface pattern of target points of the cornea in such a way that, in the pretreatment of the cornea, for example by way of the intrastromal photodisruption of a region of the cornea predefined by the interface pattern, it brings about the production of at least one interface between parts of the cornea for absorbing a gas volume that arises as a result of photodisruption. The aim here is to produce a state in the cornea which corresponds to a state after a refractive pretreatment carried out a long time before, for example by application of a SMILE treatment in which the eye is refractively corrected by the production of a lenticule within the cornea and the subsequent removal thereof through a narrow opening. Furthermore, control data for the refractive correction of the eye after the pretreatment can be calculated which predefine the pattern of target points in such a way that, during the refractive correction, it causes a gas volume that arises as a result of photodisruption to be absorbed in at least one interface produced in the pretreatment.

In the method for generating control data, the control data can predefine the pattern of target points in such a way that a refractive correction of the eye treated by the pretreatment of more than 0 and less than 1.5 diopters, for example 0.01 to 1.2 diopters, in another example 0.1 to 1 diopter, is brought about. Furthermore, data about the pretreatment, for example about the interface between parts of the cornea which is produced by the pretreatment, can be accessed and the data can be used for the calculation of the control data.

The control device of the treatment apparatus can control the laser device with the control data. The control data can be calculated such that the modifications mentioned above with respect to the planning device are realized. The control data can be generated in a planning device in accordance with one of the embodiments above, a computer and/or in the control device of the treatment apparatus. The control data can be communicated to the control device of the treatment apparatus via an interface. In some embodiments, predefining at least one three-dimensional pattern of target points of the cornea includes predefining a position of the pattern in the cornea and/or predefining a shape of the pattern which bring about the refractive correction of the eye treated by the pretreatment.

The method for generating the control data can be carried out without recourse to human interaction. It can be carried out by a planning device in accordance with one of the embodiments above that is connected to the control device of the treatment apparatus in a data-conducting manner and/or is integrated therein, e.g. a computer, which planning device ascertains the control data from corresponding predefinitions, for example on the basis of data about the pretreatment. The data of the pretreatment can predefine the position of an interface between parts of the cornea that is produced in the pretreatment. For example, the interaction of a physician is in no way necessary when ascertaining the control data since no therapeutic intervention is associated with the ascertainment of the control data. The same applies to the determination of at least one three-dimensional pattern of target points of the cornea by the planning device of embodiments.

Another embodiment relates to a method for refractive correction of an eye after a pretreatment, for example after a refractive pretreatment, or for refractive correction of an eye that includes a pretreatment; for example using a treatment apparatus in accordance with one of the embodiments above; wherein a pulsed laser beam is focused on target points of the cornea of the eye and the cornea is modified by application of photodisruption; wherein at least one part of the target points is arranged in at least one three-dimensional pattern such that the intrastromal photodisruption of a region of the cornea predefined by the pattern, a change in volume of the cornea that results from said intrastromal photodisruption and a refractive correction of the eye treated by the pretreatment are effected.

In this case, the refractive correction can include the pretreatment. Furthermore, in the pretreatment at least one part of the target points can be arranged in at least one interface pattern such that the pretreatment of the cornea, for example the intrastromal photodisruption of a region of the cornea predefined by the interface pattern, is effected and at least one interface between parts of the cornea for absorbing a gas volume that arises as a result of photodisruption is produced. The aim here, if the refractive correction includes the pretreatment, is to produce a state in the cornea which corresponds to a state after a refractive pretreatment carried out a long time before, for example by application of a SMILE treatment in which the eye is refractively corrected by the production of a lenticule within the cornea and the subsequent removal thereof through a narrow opening. Furthermore, a gas volume that arises as a result of photodisruption can be guided away into the interface during the refractive correction.

The target points can be arranged in at least one three-dimensional pattern such that a refractive correction of the eye treated by the pretreatment of more than 0 and less than 1.5 diopters, for example 0.01 to 1.2 diopters, in another example 0.1 to 1 diopter, is effected.

In some embodiments, arranging the target points in at least one three-dimensional pattern includes arranging the target points in a position of the pattern in the cornea and/or arranging the target points in a shape of the pattern which bring about the refractive correction of the eye treated by the pretreatment. The pretreatment can include removal of parts of the cornea of the eye by way of producing an interface between parts of the cornea. The three-dimensional pattern can be determined by the planning device of the treatment apparatus and/or can be contained in control data generated by the method for generating the control data of embodiments. Embodiments of the method for refractive correction of an eye can include the method for generating control data for a laser device of a treatment apparatus or modifications thereof.

The shape of the pattern of target points can be predefined in the method for generating control data in accordance with embodiments and/or can be generated in the method for refractive correction of an eye in accordance with embodiments such that at least one part of the gas volume that arises as a result of the photodisruption is guided away through micro-perforations in the cornea. The pattern of target points can be predefined and/or produced in a position adjacent to or adjoining at least one interface between parts of the cornea which is produced by the pretreatment. The three-dimensional shape of the pattern of target points can be predefined and/or produced such that at least one part of the gas volume that arises as a result of the photodisruption is guided away into the interface. The interface can be produced or have been produced by a pretreatment by application of photodisruption, a refractive pretreatment, by a lenticule extraction pretreatment, a LASIK pretreatment and/or a SMILE pretreatment. Furthermore, the pattern of target points can be predefined and/or produced in a position anterior to the interface. The pattern of target points can be predefined and/or produced with a plurality of circular planes of target points arranged one over another and/or with a plurality of ring-shaped series of target points. The ring-shaped series can be arranged in the shape of a spiral. The circular planes of target points arranged one over another can be predefined and/or produced with a shape and a number which bring about the refractive correction of the eye treated by the pretreatment. The pattern can be predefined and/or produced with a plurality of ring-shaped series of target points arranged next to one another which form a first connection structure to the interface. Furthermore, the pattern can be predefined and/or produced with a plurality of ring-shaped series of target points arranged next to one another which form a second connection structure between the circular planes of target points and/or to the first connection structure to the interface. In a further modification, in order to form the first connection structure, the ring-shaped series of target points can be predefined and/or produced in an order from posterior to the interface to anterior to the interface. The plurality of circular planes of target points arranged one over another can be predefined and/or produced in an order proceeding from the plane closest to the interface. The shape and/or the number of circular planes of target points arranged one over another can be predefined and/or produced by blocking out laser pulses.

One embodiment specifies a computer program product comprising one or more program modules which, upon execution on a computer, carry out the method for generating the control data in accordance with one of the embodiments above. The planning device of embodiments can be configured as a computer. An additional embodiment relates to a computer program product comprising one or more program modules which have the effect that the treatment apparatus in accordance with one of the embodiments above carries out the steps of the method in accordance with at least one of the embodiments above, for example when the program modules are loaded into a memory unit of the treatment apparatus, e.g. into a memory unit of the planning device.

With the above-specified embodiments and modifications of the method for generating control data and of the method for refractive correction of an eye, it is possible to realize the same advantages, operating modes and functions as with the embodiments and modifications of the planning device and/or of the treatment apparatus for refractive correction of an eye, for example with identical and/or analogous features.

It goes without saying that the features mentioned above and the features yet to be explained hereinafter can be used not only in the specified combinations but also in other combinations or on their own, without departing from the scope of the present invention.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

The invention will be explained in more detail below on the basis of example embodiments, with reference being made to the appended drawings, which likewise disclose features essential to the invention. These example embodiments are only illustrative and should not be construed as restrictive. For example, a description of an example embodiment having a multiplicity of elements or components should not be construed as meaning that all of these elements or components are necessary for implementation. Rather, other example embodiments may also contain alternative elements and components, fewer elements or components, or additional elements or components. Elements or components of different example embodiments can be combined with one another, unless indicated otherwise. Modifications and variations that are described for one of the example embodiments can also be applicable to other example embodiments. In order to avoid repetition, elements that are the same or correspond to one another in different figures are denoted by the same reference signs and are not explained repeatedly. In the figures:

FIGS. 1a and 1b schematically depict one example of a treatment apparatus 10 for refractive correction of an eye after a pretreatment with one example of a planning device 100 for generating control data for a laser device;

FIG. 1c schematically depicts a basic illustration concerning the introduction of pulsed laser radiation into the eye by application of the treatment apparatus 10 from FIGS. 1a and 1b;

FIG. 1d schematically depicts a pattern of target points of the laser radiation in a lateral cross-sectional view, said pattern being determined by the planning device 100;

FIG. 2a schematically depicts a method for generating control data for a laser device of the treatment apparatus 10;

FIG. 2b schematically depicts a method for refractive correction of an eye after a pretreatment;

FIGS. 3a to 3g schematically depict, on the basis of a treated eye, a further example in lateral cross-sectional views (FIGS. 3a, 3c, 3e, 3g) and in cross-sectional views from above (FIGS. 3b, 3d, 3f);

FIG. 4 schematically depicts a modification of the example from FIGS. 3a to 3g in a lateral cross-sectional view of the treated eye.

### DETAILED DESCRIPTION

The term “position anterior to the interface” in the present case should be understood as a position between interface and outer surface of the cornea. The term “position posterior to the interface” in the present case should be understood as a position between interface and inner surface, i.e. the surface, oriented toward the body of the patient, of the cornea. The term “posterior” in embodiments and examples means situated at the back, i.e. oriented toward the inner surface of the cornea, here also referred to as deep-set. The term “anterior” in embodiments and examples means situated at the top, i.e. oriented toward the outer surface of the cornea. Furthermore, it holds true here for the description of value ranges that the indication of a wide range with narrower alternative or preferred ranges also discloses ranges which can be formed by any desired combination of indicated lower range limits with indicated upper range limits.

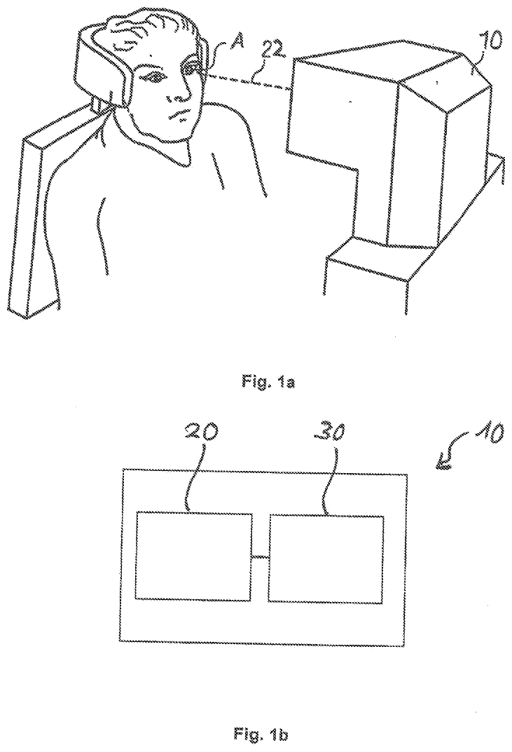

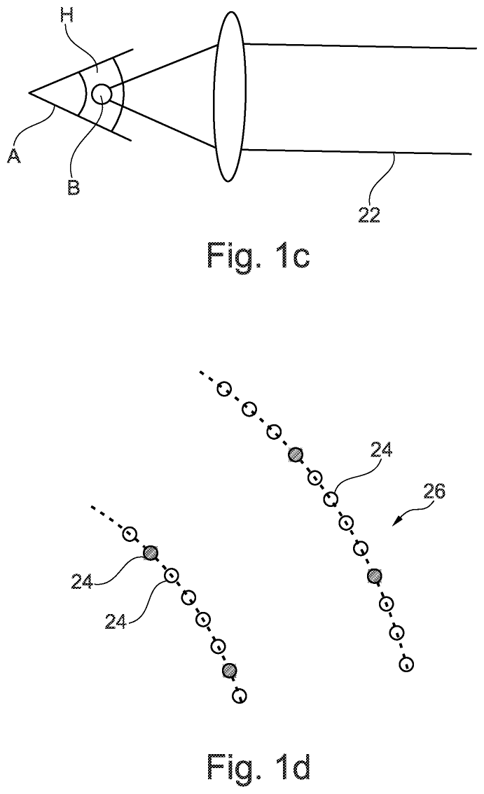

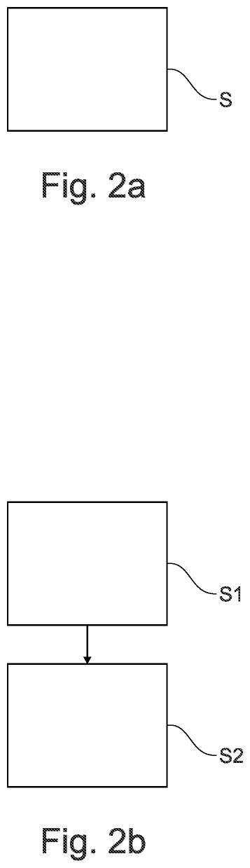

One example of a treatment apparatus 10 for refractive correction of an eye after a pretreatment is illustrated in FIGS. 1a and 1b. The treatment apparatus 10 comprises an example planning device 100 for generating control data for a laser device.

The treatment apparatus 10 is configured to introduce laser pulses at an eye A of a patient. For this purpose, the treatment apparatus 10 comprises a laser device 20, which emits a laser beam 22 that is directed in a focused manner into the eye A or the cornea H thereof, which is illustrated in FIG. 1c. The laser beam 22 is a pulsed laser beam having a wavelength of between 300 nanometers and 10 micrometers. Furthermore, the pulse length of the laser beam 22 is in the range of between 1 femtosecond and 100 nanoseconds, with pulse repetition rates of 50 kilohertz to 100 megahertz and pulse energies of between 0.01 microjoule and 0.01 millijoule being possible. The laser beam 22 is directed onto target points 24 in the region B of the cornea H. In the cornea of the eye, by way of deflection of the pulsed laser radiation, the treatment apparatus 10 produces a juxtaposition of target points 24 in a pattern 26, as is shown by way of example in FIG. 1d. For this purpose, a scanner and/or a radiation intensity modulator can be provided (not shown) in the laser device 20.

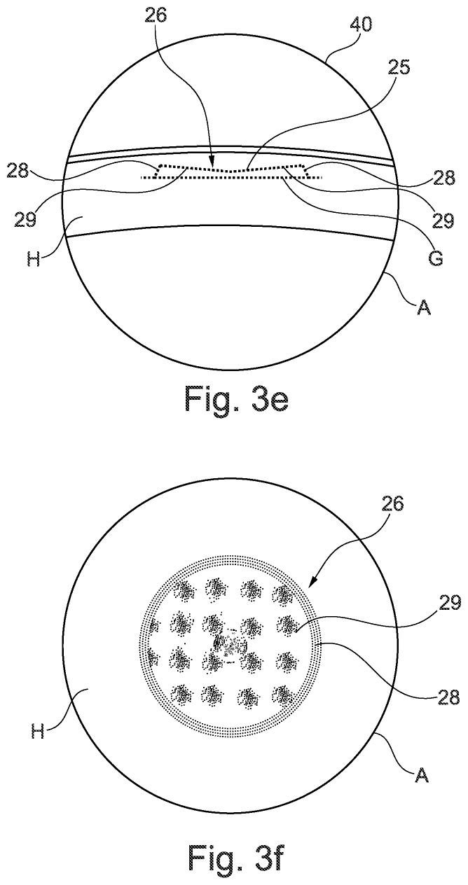

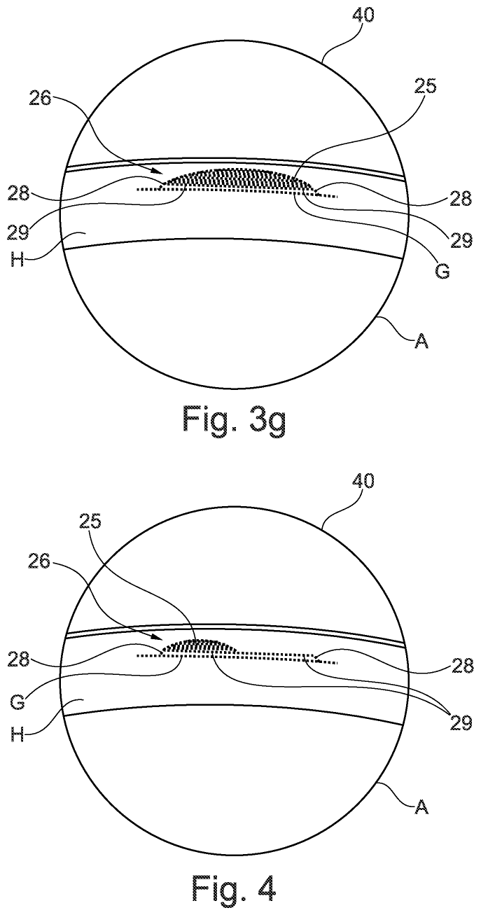

In the present example, the laser device 20 comprises an optional fixation device (not shown in FIGS. 1a to 1c), which positionally fixes the cornea H of the eye A relative to the laser beam 22. The fixation device comprises a contact glass 40 (shown in FIGS. 3a to 4), to which the cornea H is applied by negative pressure and which imparts a desired geometric shape to the cornea.

The treatment apparatus 10 has a control device 30 configured to control the laser device 20 for focusing the laser beam on the target points 24 of the cornea H and for modifying the cornea by application of photodisruption. In addition, the control device 30 can in principle control the operation of the treatment apparatus 10. For this purpose, there are suitable data connections, for example connection lines to the laser device 20. Naturally, this communication can also be implemented in different ways, for example via light guides or by radio. The control device 30 makes appropriate adjustments to and controls the timing of the treatment apparatus 10, for example the laser device 20, and hence brings about an appropriate method sequence on the treatment apparatus 10.

As shown in FIG. 1b, the planning device 100 is contained in the control device 30 in the present example. The planning device 100 is configured for generating control data for the laser device 20 and for determining a three-dimensional pattern 26 of the target points 24 of the cornea. The pattern 26 is shown in part and by way of example with series of the target points 24 in FIG. 1d. The pattern 26 brings about a refractive correction of the eye treated by the pretreatment by virtue of the intrastromal photodisruption of the region B of the cornea predefined by the pattern 26.

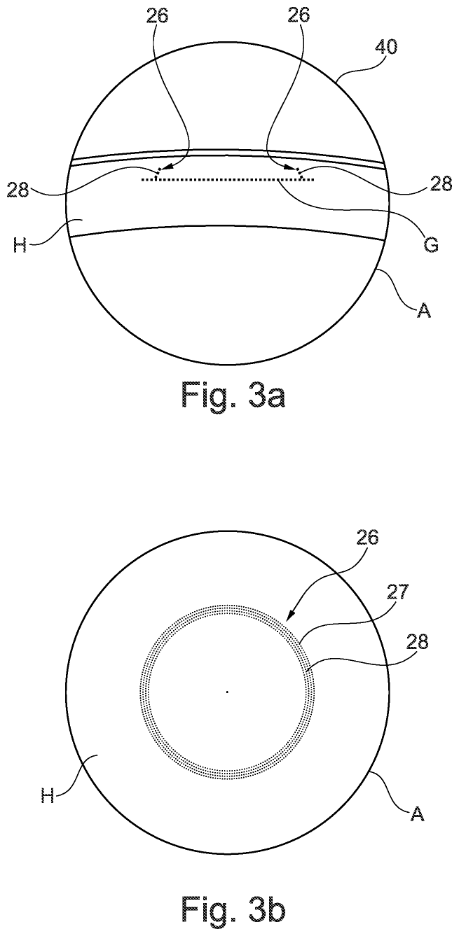

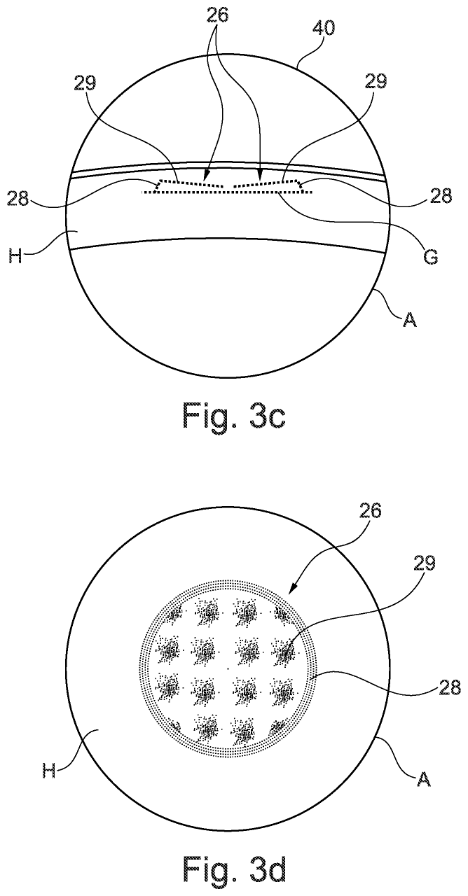

In the present example, the planning device 100 determines the three-dimensional pattern 26 by application of a method for generating control data for the laser device 20 of the treatment apparatus 10, as shown in FIG. 2a. The planning device 100 of the treatment apparatus calculates control data in a step S of the method, which predefine a three-dimensional pattern 26 of target points 24 of the cornea H. The pattern 26 is determined in such a way that a refractive correction of the eye treated by the pretreatment is obtained by virtue of the intrastromal photodisruption of the region B of the cornea predefined by the pattern 26. The pattern 26 and the refractive change in volume that is to be achieved therewith can be determined on the basis of the Munnerlyn formula. The control data are calculated on the basis of data of the pretreatment which predefine for example the position of an interface G between parts of the cornea which is produced in the pretreatment. For this purpose, the planning device can comprise an interface for receiving data about the pretreatment, for example about the interface between parts of the cornea which is produced by the pretreatment.

During an example of operation of the treatment apparatus 10, a refractive correction of the eye A is carried out after a pretreatment, wherein the pretreatment in this example is a refractive pretreatment that has already taken place previously and includes removal of parts of the cornea of the eye, such as a lenticule extraction. The method for refractive correction of the eye A after a pretreatment is illustrated in FIG. 2b. The control device 30 controls the laser device 20 with the control data calculated by the planning device 100. A pulsed laser beam is focused on target points 24 of the cornea H of the eye by the laser device 20 and the cornea is modified by [[means]] application of photodisruption, step S1. In this case, in step S2, the target points in the three-dimensional pattern 26 are arranged such that the intrastromal photodisruption of the region B of the cornea H predefined by the pattern 26 produces a refractive correction of the eye A treated by the pretreatment. In this case, the target points 26 are arranged one over another in circular planes 25. This can be effected for example by spirally scanning the laser beam 22, also called spiral scanning. The circular planes 25 of the target points 24 arranged one over another are determined and produced with a position in the cornea, a shape and a number which bring about the refractive correction of the eye A treated by the pretreatment. The circular planes 25 of target points arranged one over another are furthermore produced in an order beginning in that plane 25 of the region B which is situated at the deepest level in the eye A. In this regard, the gas that arises as a result of the photodisruption can be guided away into a layer that has already been subjected to laser treatment and thus perforated.

In a further example, the pattern 26 of target points 24 is determined in an anterior position adjacent to an interface G between parts of the cornea H, said interface having been produced by the pretreatment, by application of the planning device 100 and is produced in a corresponding method. This is illustrated in FIGS. 3a to 3g. Since the gas volume produced as a result of the laser process, i.e. the photodisruption, is guided away into the interface G, it accumulates posterior to the laser radiation and does not obstruct the laser transmission of succeeding laser pulses.

For the pattern 26, firstly a plurality of concentric, substantially ring-shaped series 27 of target points 24 arranged next to one another are determined and produced, which form a first connection structure 28 to the interface. This is shown by FIG. 3a in a lateral cross-sectional view of the treated eye A and FIG. 3b in a cross-sectional view of the treated eye A from above. The first connection structure 28 is also called an interface connector. In this case, the ring-shaped series 27 of target points 24 are determined and produced in an order from posterior to the interface G to anterior to the interface G. This can be effected by spirally scanning the laser beam 22. In the present example, the posterior end of the first connection structure 28 adjoins the interface G and the first connection structure together with the interface G forms an acute angle.

Then, as is illustrated in FIGS. 3c and 3d, concentric, substantially ring-shaped series 27 of target points 24 arranged next to one another are determined and produced, which form a second connection structure 29, which is also called a layer connector. This can likewise be effected by spirally scanning the laser beam 22. The series 27 of the second connection structure 29 are provided in an order proceeding from the anterior end of the first connection structure 28 to the center of the ring-shaped series 27 and from anterior to posterior, i.e. with a slope to the center, a region not subjected to laser treatment being left in the middle. The second connection structure 29 is connected to the first connection structure 28 in this case.

Afterward, circular planes 25 of target points 24 arranged one over another are determined and produced on the second connection structure 29, and the position, pattern and overall shape thereof, by virtue of the intrastromal photodisruption of the cornea H, produce a refractive correction of the eye A treated by the pretreatment. This is illustrated in FIGS. 3e to 3g. As shown in FIGS. 3e and 3f, this is done in an order beginning in the region not subjected to laser treatment in the middle of the second connection structure 29, i.e. in an order proceeding from the plane 25 closest to the interface G, that is to say from posterior to anterior. In this case, the circular planes 25 of the target points 24 arranged one over another are connected to one another by the second connection structure 29. Finally, the complete pattern 26 shown in FIG. 3g is obtained, which is in the nature of an example for myopia correction of the eye.

In the present example, the three-dimensional shape of the pattern 26 of target points 24 is determined and produced such that at least one part of the gas volume that arises as a result of the photodisruption is guided away via the second connection structure 29 and the first connection structure 28 into the interface G. The guiding away of the gas that arises during the laser treatment to deeper, i.e. posterior, layers and in the present case to the interface G is fostered on account of the lower level of corneal collagen crosslinking in the posterior region of the cornea H. This guiding away of the gas that arises prevents an obstruction of the laser radiation of the subsequent laser pulses and an accumulation of gas centrally in the patient's cornea, e.g. in the visual axis, and promotes visual recovery. The positioning of the photodisruption locations that is required for the necessary change in the refractive power is effected by the layering of circular disruption planes, e.g. by spiral scanning. Varying the shape and number of the disruption planes makes it possible to bring about a locally varying change in curvature and therefore refractive power for the cornea. The laser treatment starts in the deepest plane of the region to be subjected to laser treatment for the refractive correction, in order that the gas that arises is guided away into the layer of the eye A that has already been subjected to laser treatment and thus perforated.

In a modification of this example, by application of additional laser pulses, it is possible to produce micro-perforations of the cornea H outwardly toward the contact glass 40 in order to support the guided gas draining of the photodisruption products, although this is optional since the interface G has a high absorption capacity for the photodisruption gas.

By way of targeted blocking out of laser pulses during the laser treatment by application of intrastromal photodisruption, e.g. using an AOM (acousto-optic modulator), it is possible to attain various asymmetrical planes in the pattern 26 and thus different laser-treated overall profiles of the cornea H, the effect of which allows not only sphero-cylindrical corrections but also further refractive power errors, e.g. higher orders of aberrations or topographical irregularities. The pattern 26 illustrated in FIG. 4 is produced by application of targeted blocking out of laser pulses and is in the nature of an example for an asymmetrical correction of higher refractive power errors.

In modified examples of the operation of the treatment apparatus 10, the refractive correction of the eye A includes a pretreatment, in which the interface G illustrated in FIGS. 3a to 4 is produced by application of photodisruption before the refractive correction that forms the pattern 26 in the examples above. In this case, in addition, at least one interface pattern of target points of the cornea H is determined by the planning device 100 and the control device 30 controls the laser device 20 with corresponding control data. The interface G between parts of the cornea is formed by way of the intrastromal photodisruption of a region of the cornea H predefined by the interface pattern. During the subsequent refractive correction of the eye, the interface G serves to absorb a gas volume that arises as a result of photodisruption.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1.-17. (canceled)

18. A computerized planning device that generates control data for a laser device of a treatment apparatus that facilitates refractive correction of an eye after a refractive pretreatment, or after refractive correction of an eye that includes a pretreatment;
wherein the pretreatment includes removal of parts of the cornea of the eye by way of producing at least one interface between parts of the cornea;
wherein the laser device is configured to modify the cornea of the eye by radiating in a pulsed laser beam and the treatment apparatus comprises a control device that controls the laser device; and
wherein the control device is configured to control the laser device to focus the laser beam on target points of the cornea and to modify the cornea by application of photodisruption;
wherein:
the planning device is configured to determine at least one three-dimensional pattern of target points of the cornea for a refractive correction of the eye treated by the pretreatment by the radiating of the pulsed laser beam to cause intrastromal photodisruption of a region of the cornea predefined by the pattern of target points thereby causing a change in volume of the cornea.

19. The planning device as claimed in claim 18:
wherein the planning device is further configured to determine, for the refractive correction of the eye that includes the pretreatment, at least one interface pattern of target points of the cornea which, in the pretreatment of the cornea, brings about the production of at least one interface between parts of the cornea that absorbs a gas volume that arises as a result of photodisruption; or
wherein the planning device is configured to determine, for the refractive correction of the eye after the pretreatment, the pattern of target points in such a way that, during the refractive correction, the pattern of target points causes a gas volume that arises as a result of photodisruption to be absorbed in at least one interface produced in the pretreatment.

20. The planning device as claimed in claim 18:
wherein the planning device is configured to determine the pattern of target points in such a way that brings about a refractive correction of the eye treated by the pretreatment of more than 0 and less than 1.5 diopters; or
wherein the planning device is configured to determine a three-dimensional shape of the pattern of target points which causes at least a portion of a gas volume that arises as a result of the photodisruption to be guided away through micro-perforations in the cornea.

21. The planning device as claimed in claim 18:
wherein the planning device is configured to determine the pattern of target points in a position adjacent to or adjoining the interface between parts of the cornea which is produced by the pretreatment; or
wherein the planning device is configured to determine a three-dimensional shape of the pattern of target points which causes at least one part of a gas volume that arises as a result of the photodisruption to be guided away into the interface; or
wherein the planning device comprises an interface for receiving data about the interface between parts of the cornea which is produced by the pretreatment.

22. The planning device as claimed in claim 18:
wherein the interface is produced by a pretreatment by application of photodisruption, a refractive pretreatment, by a lenticule extraction pretreatment, a LASIK pretreatment or a SMILE pretreatment; or
wherein the planning device is configured to determine the pattern of target points in a position anterior to the interface.

23. The planning device as claimed in claim 18:
wherein the planning device is configured to determine the pattern of target points with a plurality of circular planes of target points arranged one over another or with a plurality of ring-shaped series of target points.

24. The planning device as claimed in claim 23:
wherein the planning device is configured to determine the circular planes of target points arranged one over another with a position in the cornea, a shape or a number of which bring about the refractive correction of the eye treated by the pretreatment; or
wherein the planning device is configured to determine the pattern with a plurality of ring-shaped series of target points arranged next to one another which form a first connection structure to the interface; or
wherein the planning device is configured to determine the pattern with a plurality of ring-shaped series of target points arranged next to one another which form a second connection structure between the circular planes of target points or to the first connection structure to the interface.

25. The planning device as claimed in claim 23:
wherein the planning device is configured to determine the control data for the laser device in such a way that, in order to form the first connection structure, the ring-shaped series of target points are produced in an order from posterior to the interface to anterior to the interface; or
wherein the planning device is configured to determine the control data for the laser device in such a way that the plurality of circular planes of target points arranged one over another are produced in an order proceeding from the plane closest to the interface; or
wherein the planning device is configured to determine the control data for the laser device in such a way that the position, the shape or the number of circular planes of target points arranged one over another are produced by blocking out laser pulses.

26. A treatment apparatus for refractive correction of an eye after a refractive pretreatment, or for refractive correction of an eye that includes a pretreatment;
wherein the pretreatment includes removal of parts of the cornea of the eye by way of producing at least one interface between parts of the cornea;
wherein the treatment apparatus comprises a laser device that modifies the cornea of the eye by radiating in a pulsed laser beam and a control device that controls the laser device; and wherein the control device is configured to control the laser device to focus the laser beam on target points of the cornea and to modify the cornea by application of photodisruption;
wherein:
the control device comprises the computerized planning device as claimed in claim 18.

27. A method of generating control data for a laser device of a treatment apparatus for refractive correction of an eye after a refractive pretreatment, or for refractive correction of an eye that includes a pretreatment;
wherein the pretreatment includes removal of parts of the cornea of the eye by way of producing at least one interface between parts of the cornea;
wherein the laser device modifies the cornea of the eye by radiating in a pulsed laser beam, focuses the laser beam on target points of the cornea and modifies the cornea by application of photodisruption;
the method comprising:
calculating control data for the laser device which predefine at least one three-dimensional pattern of target points of the cornea for a refractive correction of the eye treated by the pretreatment by radiating the pulsed laser beam to cause intrastromal photodisruption of a region of the cornea predefined by the pattern of target points thereby causing a change in volume of the cornea.

28. The method as claimed in claim 27:
further comprising calculating control data for the refractive correction of the eye that includes the pretreatment which predefine at least one interface pattern of target points of the cornea in such a way that, in the pretreatment of the cornea, the at least one interface pattern brings about the production of at least one interface between parts of the cornea that absorb a gas volume that arises as a result of photodisruption; or
further comprising calculating control data for the refractive correction of the eye after the pretreatment which predefine the pattern of target points in such a way that, during the refractive correction, the gas volume that arises as a result of photodisruption is absorbed in at least one interface produced in the pretreatment; or
further comprising calculating the control data to predefine the pattern of target points in such a way that a refractive correction of the eye treated by the pretreatment of more than 0 and less than 1.5 diopters is brought about; or
further comprising accessing data about the pretreatment, including the interface between parts of the cornea which is produced by the pretreatment, and using the data for the calculation of the control data.

29. A method of refractive correction of an eye after a refractive pretreatment, or for refractive correction of an eye that includes a pretreatment;
wherein the pretreatment includes removing parts of the cornea of the eye by producing at least one interface between the parts of the cornea;
using a treatment apparatus as claimed in claim 26;
focusing a pulsed laser beam on target points of the cornea of the eye and modifying the cornea by application of photodisruption;
wherein:
at least one part of the target points is arranged in at least one three-dimensional pattern for a refractive correction of the eye treated by the pretreatment, and wherein the at least one three-dimensional pattern predefines the intrastromal photodisruption of a region of the cornea and a change in volume of the cornea results from said intrastromal photodisruption.

30. The method as claimed in claim 29:
wherein the refractive correction includes the pretreatment, or
wherein in the pretreatment at least one part of the target points is arranged in at least one interface pattern such that the pretreatment of the cornea is effected and at least one interface between parts of the cornea that absorbs a gas volume that arises as a result of photodisruption is produced; or
wherein a gas volume that arises as a result of photodisruption is guided away into the interface during the refractive correction.

31. The method as claimed in claim 29, further comprising arranging the target points in at least one three-dimensional pattern such that a refractive correction of the eye treated by the pretreatment of more than 0 and less than 1.5 diopters is effected.

32. A non-transitory computer program product that is not a carrier wave or signal comprising one or more program modules which, upon execution on a computer, carry out the method as claimed in claim 10.

33. A non-transitory computer program product that is not a carrier wave or signal comprising one or more program modules which have the effect that a treatment apparatus for refractive correction of an eye after a refractive pretreatment, or for refractive correction of an eye that includes a pretreatment;
wherein the pretreatment includes removal of parts of the cornea of the eye by way of producing at least one interface between parts of the cornea;
wherein the treatment apparatus comprises a laser device that modifies the cornea of the eye by radiating in a pulsed laser beam and a control device that controls the laser device; and wherein the control device is configured to control the laser device to focus the laser beam on target points of the cornea and to modify the cornea by application of photodisruption carries out the steps of the method as claimed in claim 27, when the program modules are loaded into a memory unit of the treatment apparatus.

34. A non-transitory computer program product that is not a carrier wave or signal comprising one or more program modules which have the effect that a treatment apparatus for refractive correction of an eye after a refractive pretreatment, or for refractive correction of an eye that includes a pretreatment;
wherein the pretreatment includes removal of parts of the cornea of the eye by way of producing at least one interface between parts of the cornea;
wherein the treatment apparatus comprises a laser device that modifies the cornea of the eye by radiating in a pulsed laser beam and a control device that controls the laser device; and wherein the control device is configured to control the laser device to focus the laser beam on target points of the cornea and to modify the cornea by application of photodisruption carries out the steps of the method as claimed in claim 29, when the program modules are loaded into a memory unit of the treatment apparatus.
